(19) United States
(12) Patent Application Publication
(10) Pub. No.: US 2025/0054634 A1
(43) Pub. Date: Feb. 13, 2025

(54) Self-Controlling Wearable Device

(71) Applicant: LifeQ B.V., Amsterdam (NL)

(72) Inventors: Laurence Richard Olivier, Alpharetta, GA (US); Etienne Terblanche, Alpharetta, GA (US); Johan Gerard Jacobs, Stellenbosch (ZA); Franc Paul, Den Dolder (NL)

(21) Appl. No.: 18/716,788

(22) PCT Filed: Dec. 12, 2022
(86) PCT No.: PCT/US2022/081347

§ 371 Date: Jun. 5, 2024

Related U.S. Application Data
(60) Provisional application No. 63/288,300, filed on Dec. 10, 2021.

(51) Int. Cl. G16H 50/30; G16H 40/67; H04W 4/38; H04W 4/80

(52) U.S. Cl. CPC G16H 50/30; G16H 40/67; H04W 4/38; H04W 4/80

(57) ABSTRACT

A system for non-invasive health monitoring in a battery efficient and continuous manner. The system including at least one non-invasive wearable device capable of collecting and storing data. The wearable device is configured to monitor the health of a user, analyze physiological data of the user, and send data directly to the cloud.

### CROSS-REFERENCE TO RELATED APPLICATIONS

This patent application claims the benefit of U.S. Provisional Application No. 63/288,300 filed Dec. 10, 2021, the entirety of which is incorporated by reference herein.

### FIELD OF THE INVENTION

The present invention is related to the field of non-invasive digital health monitoring, physiological signal processing, and computation of biological data.

### BACKGROUND OF THE INVENTION

The health benefit derived from tracking your biometrics over time is gaining traction in the consumer wearable space as well as with clinical professionals. Consumers now use a wearable device daily that can track their health with one of the many devices being offered on the market. Such wearable devices, however, are only capable of limited functionality when functioning independently and rely heavily on external edge computing devices such as cell phones to perform a majority of computing and data exchange tasks. On the other hand, devices that both retrieve and analyze data internally are battery heavy, slow, and not fit for continuous health monitoring over a long period of time.

In clinical settings, patient monitoring is generally intrusive, expensive, and frequently limited to the time that the patient is in a hospital ward or a healthcare facility. Apart from available wearable devices already on the market, which collect a very narrow range of biometric data, there is very little available on the market for measuring a person's physiology without the use of a human or an invasive device.

Therefore, there is a need for a more robust wearable device capable of monitoring and collecting a large range of biometric data of an individual in a non-invasive way.

### SUMMARY OF THE INVENTION

The claimed invention aims to provide systems and methods for non-invasive health monitoring in a battery efficient and continuous manner. In an aspect, the system is configured to continuously monitor the physiological state of a user through the use of a cloud configured to monitor and analyze physiological data related to the user and a non-invasive instrument functioning as a data acquisition device for use by the user. The device can include a power source, at least one sensor, wireless communication means configured to communicate with the cloud, memory, and at least one processor. The processor can be configured to determine at least one biological metric from data collected by the sensor, communicate the at least one biological metric to the cloud, and determine when to send the at least one biological metric to the cloud based upon a state of the power source and the nature of the at least one biological metric. In addition, the cloud can be configured to process the at least one biological metric from the data acquisition device to determine a current health state of the user.

In an aspect, the non-invasive instrument is capable of near field communication to transfer data from a first device to a second device, and is capable of communicating with a second nearby device. In addition, the system is configured to recognize the source of the sensor data, with the non-invasive device capable of determining the proximal relation between more than one sensor data relative to each other and determining the source of the sensor data on the user. In another aspect, the non-invasive device stores a user profile information that includes user information, algorithm information, and device information. In such aspects, the system can be configured to retrieve the user profile information and send the data through a communications channel, with the system determining the best communications channel for a particular communication based on one or more metric, with the metrics including, but not limited to, the state of the device, battery power available on the device, battery power required to complete the communication, processing power of the device, and processing power required to complete the communication. The algorithm information can also be configured to allow the device to filter through the data to be sent and collected (summarizing and compressing), further reducing battery consumption and ensuring continuity.

### DESCRIPTION OF DRAWINGS

FIG. 1 is a wearable device according to the prior art.

FIG. 2 is an overview of using the wearable device in a clinical setting according to an aspect of the present invention.

FIG. 3 is a wearable device according to an aspect of the present invention.

FIG. 4 is a schematic representation of different triggers for data to be sent.

FIG. 5 is a schematic representation of transferring user information from one wearable device to another.

### DETAILED DESCRIPTION

Embodiments of the invention will be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Various changes, modifications, and equivalents of the methods, apparatuses, and/or systems described herein will be apparent to one of ordinary skill in the art. The sequences described herein are merely examples and are not limited to those set forth herein and may be changed as will be apparent to one of ordinary skill in the art.

In the following description, numerous specific details are set forth. However, it is to be understood that embodiments of the invention may be practiced without these specific details. In other instances, well-known methods, structures and techniques have been shown in detail in order not to obscure an understanding of this description. However, description of functions and constructions that are well known to one of ordinary skill in the art may be omitted for increased clarity and conciseness.

### Definitions

Core—the server where the wearable data streams and biometrics are aggregated and processed into health insights.

Consulate—a standardized interface/software that can be integrated in the cloud of any company that works with wearable devices and which can bi-directionally communicate between the wearables and a server.

Communication stack—Also known as protocol stack. The stack is the software implementation of a layered set of communication protocols which work together to provide a set of network functions.

In an aspect, the wearable device is configured to maintain real-time state of the user's biometrics, a user's physiological state (e.g. sedentary, light activity, exercise, sleep, etc.), as well as events that may require immediate attention by another (e.g. a fall, an out-of-range biometric like heart rate or breathing rate). These states and data are transmitted via communication means available to the wearable device. In addition, the information generated by the device, from low urgency long time period data sets for a sleep session, to high urgency short events like a possible fall event, is allocated additional metadata like the urgency and maximum useful life of the data. However, each communications channel/option of a wearable device (e.g. Bluetooth, WiFi, Cellular) has certain costs associated in their use. These costs include: (a) how much power is consumed while the channel is in use which directly affects battery consumption, (b) the throughput of the channel (e.g., how many bytes of information per second), (c) actual monetary costs (e.g., cellular data plan costs).

The wearable device takes the state information, communications channel power consumption and cost, as well as the data urgency and shelf life together to assess how quickly the information needs to be transmitted of device and what the appropriate communications channel would be to use. For example, the device detects a possible fall. The data needed to classify the fall is modest, and the urgency is high. The device will utilize any available communications channel that would guarantee immediate delivery, regardless the impact on power consumption or data cost. In another example, the wearable device finishes filling up a fixed size file that will contain standard longitudinal biometrics that will be aggregated and summarized on a 24-hour basis. The data has a long shelf life and low urgency. The device will avoid using a higher cost communications channel (like cellular) even if no other options are available until another option like Bluetooth or WiFi becomes available. These and other aspects are disclosed below.

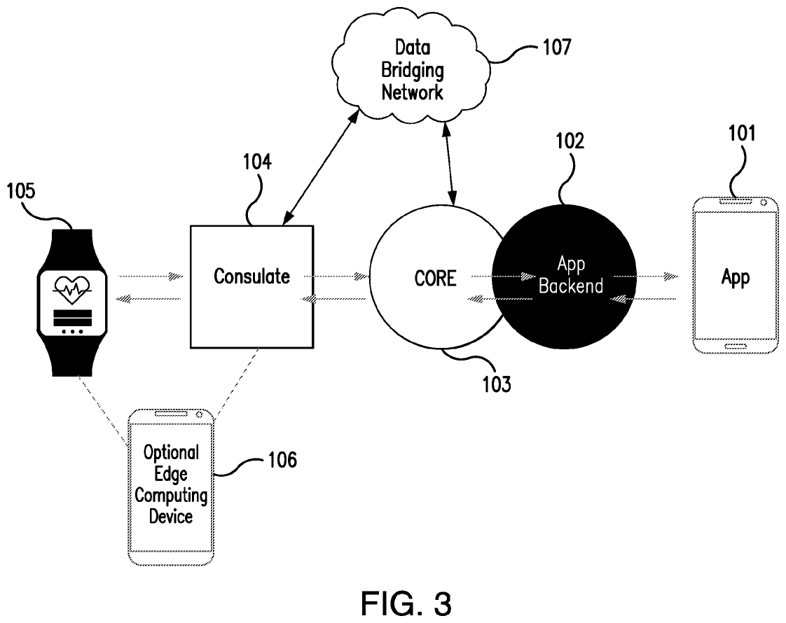

An embodiment of the claimed invention comprises a system 10 for continuously monitoring the physiological state of a user that includes a significantly improved wearable device 105, as shown in FIG. 3. The wearable device 105 is capable of controlling data exchange with a cloud (e.g., core 103/application backend 102) and behavior internally on the wearable device 105 itself. In an aspect, the wearable device 105 includes a power source (e.g., rechargeable battery), sensors (e.g., PPG, accelerometers, etc.,) memory, wireless communication means (e.g., Wi-Fi, Bluetooth, NFC, etc.), and processors. The wearable device 105 can be configured to determine biological metrics of the user from data collected by the sensors. In an aspect, the wearable devices 105 can be configured to capture and derive physiological data from a user in manners similar to those disclosed in U.S. Pat. No. 9,820,656 (issued Nov. 21, 2017); U.S. Pat. No. 11,129,568 (issued Sep. 28, 2021); and 11,291,392 (issued Apr. 5, 2022), the entirety of which are incorporated by reference herein.

The wearable device 105 utilizes an on-device configurable scheduler to transfer the data between the device 105 and the cloud 102/103. The wearable device 105 has real-time access to a user's physiological data and can make sure, by utilizing a set of pre-programmed rules downloaded to the device from the cloud or learned by the device 105, that the data is being analyzed and sent to the correct location while it is still relevant. The decisions made on the wearable device 105 can be determined by rules that are both pre-programmed or learned by the device.

In an aspect, the scheduler is as simple as a customer needing a Web portal dashboard updated with biometric information every 15 minutes, except when the person's biometrics deviate outside normative ranges for the person, in which case we want a new measurement every second (1 Hz). The scheduler will be configured by instruction from the cloud with an entry that tells it to transmit all data collected on device every 15 min (on the quarter hour), monitor biometrics against the specific user's normative ranges, and transmit 1 Hz data immediately when the biometrics go outside the normative ranges. There can and will be multiple entries in the scheduler for various types of data and specific events or states, all of which can be remotely updated/deleted/added from the cloud to the device, thus changing the scheduler's behaviour.

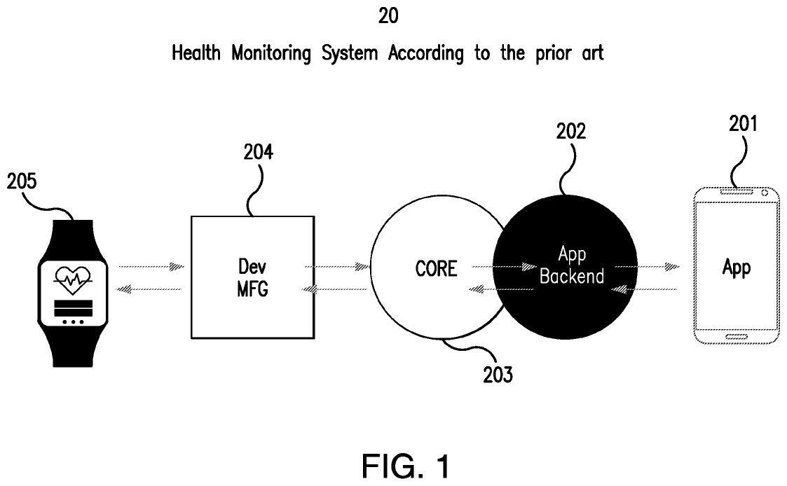

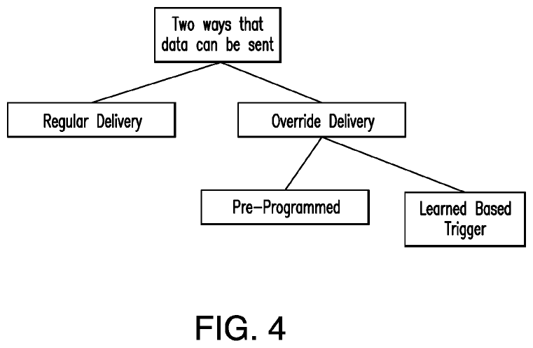

In prior art systems, external devices or other applications determine when to call to wearable devices to acquire data (see FIG. 1). According to an aspect of the present invention, the wearable device 105 is configured to support a series of communication stacks, as well as determining which communication stack to use at a given time by utilizing a set of pre-programmed rules downloaded to the device from the cloud, or learned by the device. Therefore, instead of waking up an application on an external device 106 or relying on another application to drive the data flow, the wearable device 105 can determine which data needs to be sent and how it will be communicated (see FIG. 4).

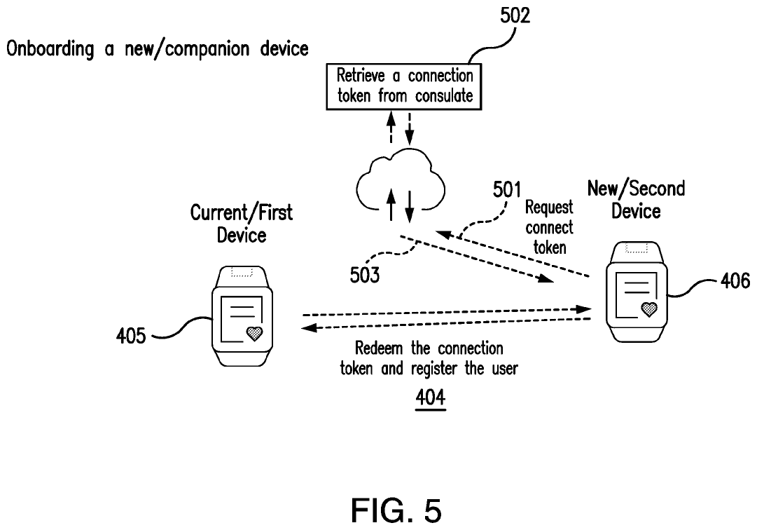

The wearable device 105 is configured to maintain user states (i.e., physiological state, e.g., sedentary, light movement, exercise, sleep, etc.) in an internal memory as well as the cloud (core 103/application backend 102) which allows for continuous data collection while transferring between devices (see FIG. 5), charging battery, or performing software updates. This is done by creating a user profile of information that is stored locally on the wearable device which further allows the device to store data until it can/should be communicated.

While maintaining confidentiality of personal identifiable information (PII), via a data bridging network 107 (FIG. 3) discussed in detail below, the wearable device 105 is able to associate users, via a user profile, with a data source (the device(s) associated with a user, linked by a user profile) which is easily accessible and transferable, allowing the wearable devices 105 to be exchanged between multiple users. Further, the ability to maintain confidentiality of PII allows for use and connections within the system 10 between different devices 405 and 406 (FIG. 5) not necessarily stemming from the same company. This also allows for use of more than one wearable device (at the same time or in the alternative) that can continuously collect data from a single user. The ability for use of multiple devices, including at the same time, allows for continuity as well as distributed sensing. If two devices are collecting information about a single user at the same time from different locations on the user's body, then the device is capable of achieving much more in terms of data analysis and health determination/prediction by dynamically selecting sensor data from the most optimal location available. Further, the device(s) can be configured to communicate with other sensor devices and retrieve data from such sources. The ability to wirelessly communicate and locate nearby devices allows the devices to contact trace between themselves, other wearable devices nearby, and other machines that are capable of communicating with and/or triangulating the location of the device.

The ability of the device 105/405/406 to both collect data and run algorithms allows the device to filter (i.e., summarize and/or compress high-resolution data in the devices) through the data to be sent and collected, further reducing battery consumption and ensuring continuity. By summarizing/compressing the data into more compact yet still relevant summaries of the data, less data is being sent, thus a smaller and briefer transmission window is needed, requiring less power. This also increases accuracy by giving the device the capability to determine which analysis is done and where. The various unique capabilities of the claimed invention are described in detail below.

Intelligent Communication with the Application

Most wearable devices 205 of prior art systems 20 transfer data by cloud-to-app push notifications. These notifications wake up an app on a user's smartphone 201, with the backend/cloud 202/203 pulling data from a wearable device 205 using bluetooth or bluetooth low energy (BLE) (See FIG. 1). The wearable device 105 itself of the present invention 10 (see FIG. 3) controls data exchange/flows and its behavior without the use of a push notification. The device 105 runs a configurable scheduler to initiate transfers from device 105 to the core 103/application backend 102/cloud. This allows for a more power efficient use of communications stacks (like bluetooth or BLE), but could include higher power options like Wi-Fi and allows the device 105 to utilize higher throughput data transfer. The device 105 uses the real-time contextual knowledge and capability to run logic to make those decisions. The device 105 has a routing engine that follows a set of configurable rules based on the connectivity options, and their costs as discussed above, available to the wireless device 105 (e.g., only transfer data when you are in a certain geographic area, transfer data via Wi-Fi when Wi-Fi at a certain bandwidth is detected, etc.). This can only be done if the logic is being done on the device 105 and not in the cloud.

Using on-device algorithms, the wearable device 105 can create rulesets to override the scheduler and push data from the device 105 to the cloud 102/103 when needed. For example, the device 105 can utilize the scheduler until a health emergency of the user occurs. As the wearable device 105 is configured to monitor physiological parameters of an individual (e.g., heart rate, breathing rate, SpO, etc.), the wearable device 105 can determine normal physiological parameters for the user, and send information when the wearable device 105 detects any deviation from those ranges. For example, the device 105 is configured to learn through observation that a person's normal breathing rate falls between X and Y. As long as real-time observations are in this range, the scheduler is used. However, if the breathing rate goes out of the normative range, there is a scheduler override to fast track sending the data. In other words, the wearable device 105 can be configured to send data as ordered by the scheduler until some unexpected event tied to the wearer of the wearable device 105 occurs. Other examples include a fall detector, blood oxygen detector, panic button, user moves into a dangerous geo-fenced area, etc.

This method of initiating transfers from a device 105 is crucial because activation of wireless communication is a major contributor to battery consumption. Live streaming data drains the battery, which is counter-intuitive to a product that is intended to work continuously. Therefore, the present invention works on a scheduler so that it doesn't drain the battery. By doing so, the system 10 also eliminates the need to utilize push notifications. However, as shown in FIG. 4, the system is flexible: it is not configured only to be able to receive information when the scheduler is triggered—the scheduler can be overwritten to push forward data when there is an anomaly or a predetermined outlier that the device is searching for. The system 10 uses real-time contextual knowledge to run the process on the device 105; the device 105 itself is then able to determine when to send over information—that is, stick to a schedule as determined by a scheduler, or to send information when needed (e.g., an alert, etc.). The thresholds/anomalies can be determined by the device 105 itself. In other aspects, the device 105 can follow rules pre-set that say to send the data when a threshold is met or an anomaly is detected, but leaves it up to the device 105 to determine what is the threshold/anomaly. If a physiological measurement triggers an anomaly or outlier, the device will send the data outside of the normal scheduler. In this way, the device has both a regular delivery and an “override” delivery. The device is capable of using any physiological measure/combination to determine the state that would trigger an override. Further, the device might utilize non-physiological states that it is capable of collecting such as biometrics, quality control, positioning of the device, etc.

To generalize, the device 105 has real-time access to a whole series of information, including, but not limited to, physiological data associated with the wearer of the device, and the status of the device itself (e.g., battery state, memory status, storage space, etc.). The system 10 can provide to the device 105 a rule and making provisions on a per-device basis to tell the device to change the rules for reporting and scheduling. For example, the system 10 could be utilized by a mining company that requires specific rules, such as when to override the scheduler when an anomaly related to oxygen saturation levels occurs, or if the battery is below a certain percentage then don't send data. In a fragile care facility, rules could utilize a fall detector, and when the fall detector detects a fall, then the device should push forward the data so a monitoring center can analyze and respond to the fall of the wearer of the device. The key here is flexibility: a device 105 that can be updated with modified rules that determine how to decide when to push forward data based on the specific needs and desires of the user. The reason that this is so important is because some data has a certain “shelf-life.” For example, when someone's heart stops, it is not useful to be notified of this change 12 hours later when the device 105 is connected to an external power source. Therefore, the goal in creating these overrides is to ensure that critical data is transferred in real-time, to the correct location, while it still has use. All the while, the device 105 is preserving the battery life by only sending data that is less time-sensitive using a scheduler.

In summary, data is sent in two cases: First, when it is scheduled to be sent and all data is shared (“regular delivery”), or second, when there is an override and specified data is sent to a specified location (“override delivery”). These overrides can be pre-programmed (by the user, beneficiary of the information, hospital, etc.) or it can be determined by the device itself (learning-based trigger). A dynamic trigger is determined by the application as it learns the behavior and physiology of the user.

### Intelligent Communications Stack Decisions

The wearable device 105 is also configured to abstract the communications stack to provide support for multiple communications channels (such as Bluetooth, BLE, Wi-Fi, LTEm, LTE, etc.) according to an aspect of the present invention. This is extremely useful in the case where information is time-sensitive and the user does not have access to their Wi-Fi or a phone. In such instances, the device can use other available communication channel(s), including, but not limited to, LTEm. The routing function, performed by the routing engine, receives outbound and inbound data tagged by the device, based on pre-programmed rules downloaded to the device or learned by the device, with metadata indicating the urgency of the communications, max “shelf life” (how long it can be buffered/delayed before it becomes irrelevant), the needs of the downstream consumers of the data, etc. In an aspect, the metadata is configurable information that is transmitted from the cloud to the device for on-device persistence until the updated information is received, from the device to the cloud. The actual nature of the metadata (value, ranges, etc.) can be manually determined or can be calculated through some automated process. The routing engine is also configurable with a ruleset that enumerates the communication options, and for each the “cost” of such an activity such as battery impact, throughput, and other considerations. The device can dynamically decide which communications stack to use at a given time to improve speed, reduce cost, preserve battery life, etc. (e.g., disregard battery impact for immediate transmittal of a fall event data, delay using a higher cost option for low importance, long use data (e.g., data related to a sleep session) until a lower cost option becomes available).

Current wearable devices use a default approach. The system 10 and wearable device 105 of the present invention have a much more intelligent way, based on a number of considerations, to determine whether and how we send different types of data. For example, the device 105 can be configured to wait to send large data sets over Wi-Fi when it becomes available as opposed to bluetooth. The wearable device 105 can also be told to make these determinations, for example, based on battery power on the device 105. In a retail consumer space, it is assumed that each wearable device has a companion device. However, in different settings, (e.g., a hospital, fragile care facility, etc.), the assumptions are different.

Given that the wearable device 105 is configure to utilize various stacks for communication, the wearable device 105 has a number of different communication channels on which the device 105 can communicate/send information. In other words, the wearable device 105 is not limited to moving data in only a single direction. Further, the wearable device 105 has the capability to update the device with new rules dealing with communication, such as: under what circumstances it moves data, how algorithms sample, what data to sample and transport, etc. The present invention can include interfaces for routing systems to classify payload based on priority, “shelf life,” etc. The communications channels may decide the optimal transport to use based on each transport's “cost” (battery, end user data airtime, latency, throughput, etc.).

The method employed by the system 10 answers the following questions: (1) how is the decision made to send a set of data? and (2) after that decision is made, what channel should we send it through?

### On-Device Information Buffering System

Current wearable devices use a traditional local storage method which has algorithms that harvest and store information until the device 105 can communicate and send the data through. In order to dynamically control its own behavior, the wearable device 105 includes a local information buffering system on the device capable of storing, retrieving, forwarding, and syncing information on-demand. This buffering system can move information between temporary and permanent storage in a way that improves the accuracy and consistency of the device 105, especially when power is lost for the device. For example, in order to calculate an accurate basal resting heart rate, roughly 1-2 days of a specific person's heart rate data is required. An accurate basal resting heart rate enables very accurate calorie consumption calculations on a per-individual basis. However, should the device lose power while still gathering information during the initial 1-2 day learning phase, an inaccurate resting heart rate results, and also then an inaccurate calories calculation. The buffering system allows for this information (in temporary storage) to be backed up on permanent storage, and recovered should there be a power loss, thus enabling the improvement. The system and method of the present invention has expanded this to include a capability of having a stored profile. The profile is not simply the biometrics of the user but also includes algorithm information and state as well as device behavior configuration information.

For example, a pulse-waveform algorithm which uses a certain set of rules under which it harvests raw PPG data and performs a pulse waveform analysis may be modified. If the system and/or device detects something out of the ordinary or that crosses a threshold to warrant investigation, the system can send an update to the algorithm profile to expand the scope/change the parameters/etc. of the pulse waveform algorithm for a particular individual, providing the ability to change the rules regarding how the algorithms work. The standard pulse waveform algorithm requires a lower resolution raw PPG signal, and when an event of potential interest is identified the resolution of this data can be temporarily increased in order to provide the algorithm with higher fidelity inputs which in turn result in higher specificity and sensitivity of the outputs. This is useful because, using this method, firmware updates are not needed to improve or change the algorithms stored on the device. Further, such functionality supports multiple use cases. The traditional approach to multiple use cases has been having firmware builds, each with a distinct and fixed set of algorithms with fixed predetermined behavior. Now, changing the algorithm profile for a group of devices or for an individual device changes the sampling behavior for a specific application.

The profile information can be broken down into three categories: (1) human information, (2) algorithm information (e.g., configurable information that dictate the behavior and modify some of the algorithm's parameters), and (3) device information (configurable device settings (e.g., if and how the device should vibrate for specific events like battery low or degraded sensor signal quality). Having this kind of information stored locally on the device 105 and having the ability to modify the information is new in the art. This kind of information should not be stored in random access memory (RAM) because the moment you run out of battery or the device gets recycled you will lose that information. Therefore, the use of this buffering system persists the information onto non-volatile memory. As the device learns about the user, it can update the stored profile to improve accuracy. While the act of buffering is known in the art, the wearable device uses buffering in a unique way—maintaining human biometrics and configurable device settings.

The buffering system also maintains algorithm states, that is the stage/state at which the algorithm is in at a given time. In some aspects, these algorithms take a given amount of time to generate accurate results; some algorithms may require hours or days' worth of biometric data in order to converge on a final answer. For example, converging on an accurate basal resting heart rate for a specific person by observing their 1 Hz heart rate dynamics can take hours and days, as discussed above. Further, specific conditions may be necessary (e.g., sedentary conditions when the heart rate is stable) may not be available for a period of time. Therefore, it is beneficial to know where the algorithm stands on its way to deliver the result.

An example application of this is if we want to learn about a user's resting heart rate, the device can build a histogram of candidate information for the user's day. This histogram can record at what heart rate and for what duration the user is in a stable state at different times of the day. As the day progresses, the histogram is populated. It is desirable to avoid having the device run out of battery and die in the middle of the day, losing all of this information. The buffering system allows algorithms that aggregate information over time to ask the buffering system to persist/maintain its state (i.e., at the point of process and data collected/generated up until that point in time), making the buffering system useful. So, when the battery dies and the algorithms are restarted, the algorithms can retrieve stored states from the buffering system and restore the histogram to the last state it was in before the restart and continue from where it left off (in the case of resting heart rate). This is most useful for algorithms that need to learn/converge information over a period of time in order to produce a result and, therefore, it is important that the information that has been collected is not lost prior to producing the final result.

### Associating User and Data Source

In the current state of the retail consumer wearable space, there is an assumption that the same single company services the roles of registrar (access to Personal Identifiable Information), source (wearable device), transformer (party that transforms harvested information into new information and insights), and consumer (apps, portals, etc. using harvested and transformed information to engage with users) in regards to General Data Protection Regulation (GDPR). This is because current manufacturers are vertically integrated and all of the GDPR roles are performed by the same organization. However, as soon as you start looking to integrate this technology into other spaces which require specialized solutions (such as hospital dashboards, remote monitoring, research, etc.), it defies the retail consumer wearable model assumptions. Further, the traditional mapping of: user <->device <->user's phone <->device company cloud <->device company data transformation can no longer be assumed as well.

The present invention takes a different approach. Decoupled are the registrar, source, transformer, and consumer to avoid working under the presumption that these are all the same organization. The present invention provides on-device functionality to embrace this assumption. Thus, we can separate the data from the personally identifiable information (PII).

This is particularly helpful when the information must remain private to follow privacy and consent regulations (such as HIPAA guidelines). A Data Bridge Network (DBN) 107 (see FIG. 3) is an example of a de-identification process that works as an intermediary between the wearable device 105 and the cloud/core/app backend 102/103 and separates the data and creates unique identifiers for the user and for the application accessing the information. If the system is compromised or breached, the data is anonymous and cannot be tied back to a specific user. The DBN 107 registers each new device 105 as a new “data source” and gives the source a unique identification number. When a different party consumes this information, it is transferred with a new unique identifier. A transformer (e.g., the LifeQ® core) will not accept any information with PII and has no place to store such information. The DBN 107 simply facilitates the connection between the two parties and the data flow and does not store information itself. In an aspect, the DBN 107 can operate as the DBN disclosed in U.S. Pat. No. 10,749,844, herein incorporated by reference in its entirety.

This method also provides the ability to easily onboard a new user and associate/disassociate that user to a particular device/data source, as shown in FIG. 5. There is a companion device 406 associated with a wearable device 405, there are two potential places from where to harvest information and being able to dynamically change the data source. This is also applicable in associating a user with a new device or when using more than one wearable device at a given time. In the current state of the art, a single user is “tied” to a single device (e.g., John's Airpods are associated with John). This new method allows a device 405 to request a connect token 501 from a consulate system 502 (see also FIG. 3) in order to associate itself with a new user. Consulate 104 then proxies with the DBN 107 to get a short-lived connection code 503, the device 406 redeems the connection code 404 via a consulate proxy to the DBN, and registers the user, the following are two main uses for this user association method:

### (a) Companion Device

A user can use more than one wearable device 105 at a time and there is a “preference” as to which device 105 is doing most of the work at a given time (this can be based on many factors such as battery life, type of work to be performed, etc.). The preference is based on a predetermined rule.

### (b) Swapping Out Devices

Data is continuously flowing using this new method of transferring user data. However, note that the companion device can become a swapping device, for example there is one wearable that you wear at night while you sleep and another that you wear during the day.

The functionality/utility of using multiple devices is expanded on below. A master copy of the profile information maintained by the on-device buffering system is saved in the cloud, and is transferred to any new device associated with that user. In this way, it is easy to switch the device on a given user without losing the profile information relevant to the specific user. This is unique compared to other wearable devices currently on the market because the other devices don't have the flexibility to associate a new device with a new user so quickly and seamlessly without losing profile information. Note that the DBN is used here to ensure that the device/source does not have access to the user's information during the data source change; the profile information is stored in connection with a user. This allows the device to associate to a new user without requiring personal identifiable information.

### Distributed Sensing

This wearable device 105 is not limited to a particular area of the body. Some examples of where the wearable may be worn includes: on the wrist, upper arm, various locations on torso, face (eyewear with access to temples, ear-ware with access to in-ear area), etc. The device 105 may be combined with a variety of straps, vests, and other clothing pieces with pre-sewn insert locations, clip-on/magnetized housings to attach to compression vests, bras, pants/trousers etc. The device 105 is configured to mechanically/electrically identify the location of the device on the body based on where/how the device is paired with a strap/harness/clip-on housing/magnet/etc.

If a device 105 can be moved between housing/attachment mechanisms, the device 105 is designed to detect that it has been incorporated into a different location and in a different way. This is critical in measuring physiological data measurements because your pulse, heart rate, movement, etc. may be detected differently in different positions. The location detection can be done using signal analysis to automatically detect the location of the device on a user. For instance, you can collect a pulse signal from somewhere other than the wrist and analyze the signal (there are existing softwares for this) to determine where the pulse is actually coming from. Then the device 105 can change its formulas based on where the device is located. This is useful because the different location and signal strength will not give the same results using the same formulas and algorithms. Therefore, the location of the device is critical to know and the impact of that location on the specific sensor signal.

In another aspect, the system can also incorporate a network of sensors associated with a user, the sensors harvesting information on a single body at a given time. In some versions of this aspect, the network can employ a disposable “remote sensor/accessory/peripheral” which performs as a remote peripheral/extension of a primary wearable device, with the primary wearable device having the largest rechargeable battery, storage, processing capability/power, and communications options. The peripheral device may have a fixed coin battery, specific sensor(s) with minimal electronics to service the sensor(s) and buffer the data, then leverages BLE/Bluetooth communications only to offload the data to the primary device.

Such a set-up can be utilized in the context of a cardiology solution. There are algorithms that can use the normal PPG signal to do an analysis and highlight the highly probable arrhythmia. However, doctors may want to be able to get a week's worth of ECG recordings in addition to the PPG data. The primary wearable device may be configured to work alongside a disposable, wearable ECG patch that can communicate with the wearable device via bluetooth. Instead of having an external product utilized with the ECG device in order to collect the data, the patch can leverage the primary wearable device as it's communication proxy and for further edge processing.

Another application of a primary/periphery system is in consultation solutions. For example, a doctor needs a stethoscope to get data about their patient in a remote setting (e.g., after an out-patient surgery). A digital stethoscope patch can be sent with or to the user that can then place on their body. The patch (periphery) communicates the digital stethoscope data to the wearable device (primary device). The doctor is able to view/communicate with the wearable device to view the data and control the metrics being recorded. In the current state of the art, an external digital device such as a stethoscope would utilize its own isolated communication method (such as being directly linked to a single machine) and is not configured to communicate with a wearable device. This new method creates a new communication proxy and allows us to use the wearable device system and connect to other communication devices.

This ability to integrate another disposable or permanent devices into the system which can communicate with the primary wearable device has many crucial benefits. Gathering ECG data, for example, can be extremely invasive and expensive; cardiologists typically ship high friction ECG Holter devices to patients to wear for days/weeks. The present invention offers a cheaper, easy to use way to access the same data remotely. Further, the ability to add assisting devices expands the functionality of the primary wearable device significantly. The wearable device is “future proofed” because when there is a new method of collecting data/sensing technology, you can simply incorporate a new device that utilizes the new method. For example, the primary wearable device can collect data from a digital scale, a blood pressure cuff, a thermometer, etc. that can communicate with the wearable device wirelessly and leverage the wearable as its proxy.

### Physiological State Based Algorithms

In the current state of the art, the standard approach in collecting physiological measurement data is to solve for an end objective and/or worst case scenario for algorithms. The system measures heart rate for downstream applications. It is not possible with other currently available devices on the market because continuous monitoring drains the battery. What the prior art devices (see FIG. 1) are doing right is measuring the heart rate at arbitrary intervals (e.g., every fifteen minutes). This information is useless in certain physiological measurements. Informative data is overlooked when using the “interval method.”

An example of the improvements of the current invention is when monitoring heart rate. In a twenty-four hour period, a majority of peoples' day is spent sleeping or doing very little movement at all. A user's activity can be broken down into three physiological states: (1) no motion (˜40-60% of the average user's day), (2) low motion (˜40% of the average user's day), and (3) high/exercise level motion (<=2% of the average user's day). The primary wearable device 105 is able to distinguish between the different states and applies a different algorithm for each physiological state that is created to optimize signal quality, coverage, and power consumption for each case. High motion is the heaviest and drains the battery the most while no motion is the lightest. This new approach provides full coverage of physiological measurement data and utilizes similar or less power consumption and greater efficiency than a duty cycling approach. The old approach turns the algorithm on and off while the present invention is constantly utilizing an algorithm but switches them out.

In an aspect, the wearable device utilizes a supervising algorithm that runs separate from the three separate heart rate algorithms. The supervisor algorithm maintains an awareness of the physiological state of the user (e.g., sedentary, light move, exercise, sleep, etc.) and switches between the three heart algorithms as the user switches states. For example, if the user transitions from light movement to sedentary, an algorithm optimized for sedentary state is activated by the supervisor algorithm. In an aspect, the supervising algorithm can take various forms and steps dependent on the nature of the algorithms it is supervising, and can be configured to monitor user states and battery states dependent upon their needs.

### Algorithm Tiering/Abstraction Breakdown

When you are dealing with more complex algorithms, the question is when can the complex processes be broken into consecutive stages. When algorithms are broken into steps the correct way allows the removal of dependency between the model of the algorithm running and where it can be run.

For example, sleep monitoring can be subdivided it into four parts: (1) Real-time sleep/wake monitoring, (2) raw feature aggregation, (3) model feature extraction, and (4) sleep architecture neural network, heuristic model. In the standard/traditional approach the data is harvested on the device, sent to the cloud, and then pulled apart in the cloud. However, if broken into four parts, the more critical parts (e.g., the algorithm tasked with accurately identifying potential sleep events and the capture of data required for sleep analysis (for transmission off device for further processing) can be determined, require less battery power, etc. and run only these parts on the device. In an aspect, the determination of the break down can be pre-determined (i.e., human determined in design phase of the algorithm code as structured and deployed) or can be learned via machine learning. Further, these discrete transformations can be distributed across different processing layers on the wearable, multiple wearables, other edge devices, and/or the cloud. Such a process provides instant information by performing a function on the wearable device and getting interim readings and then subsequently doing deeper and more accurate analytics in the cloud by leveraging the benefits of more computing resources. A good example is when an individual wakes up in the middle of the night. A rough calculation (e.g., sleep quality, SpO2 levels, etc.) on the device can be performed, but with a more refined calculator in the cloud being utilized for the deeper dive. There is limited processing that can be done on a wearable device and therefore it is useful to be able to select what does which analysis where.

### Companion Device Mechanisms

The primary wearable device is further able to communicate with a companion device. The primary wearable device can be configured to collect information from a second device, such as a device that is less bulky that the user prefers to wear to bed. It is also possible to wear both devices at once; the primary device is capable of determining which of the devices should be collecting data at a given moment. This relationship allows the primary wearable device to maximize efficiency by preserving battery power when there is less “work” to do. There is a defined relationship between the primary wearable device and another device. The user is able to choose for themselves which device, or both, to wear at a given moment. One may be more accurate than the other and one may be more comfortable, etc. Both devices can perform physiological sensing. Some of the sensing capabilities could be duplicated on both devices (differences being quality, coverage, and battery consumption impact). The devices may also have their own unique non-overlapping sensing capabilities.

Some use cases for this system include:
(a) Using an alternative device, where there are overlapping capabilities (sensing, computing, storage, etc.) which is only enabled on one device with the highest ROI. Here the primary device determines which device which will be used.
(b) Where the devices are used in “relay” fashion. Meaning, a small/comfortable device is used during sleep, while the bulky device is used during the day. Together the devices provide twenty-four seven coverage and data collection. The user decides which device will be used.
(c) Where complimentary sensors on multiple wearables sample signals from optimal locations and the data streams are merged and sent to the cloud (multiple location coordinated sensing).

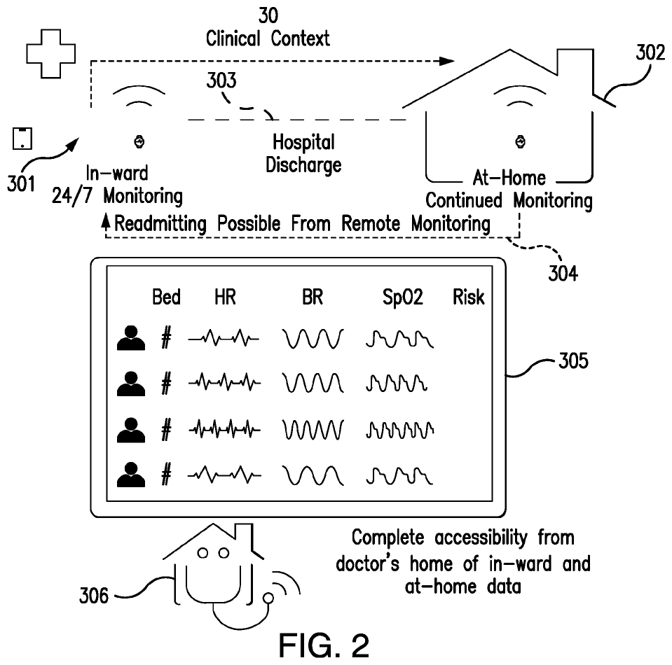

The two, or more, devices can be configured to be aware of each other using near field communication (NFC) (proximity sensing). The present invention leverages NFC capabilities that can work alongside/instead of bluetooth. If the bluetooth signal is strong, it will be utilized to listen for other wearable devices and we can measure the strength of the bluetooth signal. By being able to measure the strength of the signal, we can start to create a proximity log for specific devices to know where devices are at a given time (note that bluetooth requires less battery power than GPS). When you have all of this information in a central location, you now have contact tracing information as well as proximity location information. An example utility of this feature is in a hospital setting (See FIG. 2), we would now be able to track which nurses saw which patients at a given time.

What is claimed:

1. A system for continuously monitoring the physiological state of a user, comprising:
a. a cloud configured to monitor and analyze physiological data related to the user;
b. a non-invasive instrument functioning as a data acquisition device for use by the user comprising:
i. a power source;
ii. at least one sensor;
iii. wireless communication means configured to communicate with the cloud;
iv. memory; and
v. at least one processor, wherein the processor is configured to:
A. determine at least one biological metric from data collected by the sensor;
B. communicate the at least one biological metric to the cloud;
C. determine when to send the at least one biological metric to the cloud based upon a state of the power source and the nature of the at least one biological metric;
wherein the cloud is configured to process the at least one biological metric from the data acquisition device to determine a current health state of the user.

2. The system of claim 1, wherein determining when to send the at least one biological metric to the cloud follows a set of rules which may be pre-programmed on the non-invasive instrument or learned by the non-invasive instrument.

3. The system of claim 1, wherein the non-invasive instrument is capable of near field communication to transfer data from a first device to a second device.

4. The system of claim 1, wherein the non-invasive instrument is capable of communicating with a second nearby device.

5. The system of claim 1, wherein the system is configured to recognize the source of the sensor data.

6. The system of claim 5, wherein the non-invasive device is capable of determining the proximal relation between more than one sensor data relative to each other and determining the source of the sensor data on the user.

7. The system of claim 1, wherein the non-invasive device stores a user profile information comprising:
a. user information;
b. algorithm information; and
c. device information.

8. The system of claim 7, wherein the system retrieves the user profile information and sends the data through a communications channel, the system determines the best communications channel for a particular communication based on one or more metric, the metrics comprising:
a. state of the device;
b. battery power available on the device;
c. battery power required to complete the communication;
d. processing power of the device; and
e. processing power required to complete the communication.

9. The system of claim 7, wherein the algorithm information on the device allows the device to filter through the data to be sent and collected, further reducing battery consumption and ensuring continuity.

10. The system of claim 1, wherein the system is further configured to detect an anomaly in the user's health and directly send the data to the cloud.
